(19) United States
(12) Patent Application Publication
(10) Pub. No.: US 2003/0105048 A1
(43) Pub. Date: Jun. 5, 2003

(54) Oligobenzimidazole derivatives and their use as DNA transfecting agents

(76) Inventors: Daniel Scherman, Paris (FR); Michel Bessodes, Villejuif (FR); Bruno Pitard, Reze (FR); Javier Soto, Vigo (ES); Gerardo Byk, Qyriat Ono (IL)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/139,549

(22) Filed: May 6, 2002

Related U.S. Application Data
(63) Continuation of application No. PCT/FR00/03087, filed on Nov. 6, 2000.
(60) Provisional application No. 60/174,648, filed on Jan. 5, 2000.

(30) Foreign Application Priority Data
Nov. 5, 1999 (FR) FR 99 13934

(51) Int. Cl. C12N 15/87; A61K 48/00; C 07D 4 3/14

(52) U.S. Cl. 514/44; 435/455; 514/254.06; 514/394; 548/305.4; 544/370

(57) ABSTRACT

The invention concerns oligobenzimidazole derivatives capable of combining with nucleic acids and their uses including for transferring in vitro, in vivo, or ex vivo nucleic acids into cells or for visual display of nucleic acids administered by fluorescence.

This is a continuation of International Patent Application No. PCT/FR00/03087, filed Nov. 6, 2000 which claims the benefit of French Application No. 99/13934, filed Nov. 5, 1999 and of U.S. Provisional Application No. 60/174,648,filed Jan. 5, 2000,said application are incorporated by reference in the entireties herein.

### FIELD OF THE INVENTION

The present invention relates to oligobenzimidazole derivatives capable of combining with nucleic acids, of general formula (I):

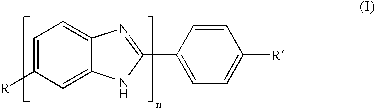

to salts thereof, to the compositions which contain them and to uses thereof, for example for the in vitro, in vivo or ex vivo transfer of nucleic acids into cells or for the visualization of the nucleic acids administered, by fluorescence.

### BACKGROUND OF THE INVENTION

Patent FR 1 519 964 describes bis-benzimidazole compounds and salts thereof, of formula:

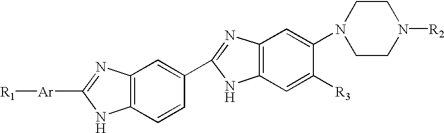

in which Ar denotes an arylene residue, R₁ denotes a hydrogen or halogen atom, a hydroxyl group, a lower alkyl or alkoxy group, a mercapto or alkylmercapto group, an alkylenedioxy or nitro group, a phenyl residue or an amino group optionally bearing alkyl substituents, R₂ denotes hydrogen, an optionally substituted alkyl residue, an alkoxycarbonyl, carbamido, aryl or aralkyl residue and R₃ denotes a halogen atom or a lower alkyl residue.

These compounds are described as having high anthelmintic and bacteriostatic activity against gram-positive microorganisms, and they can also be readily characterized by virtue of their typical green fluorescence (see page 5, paragraph 5 of FR 1 519 964).

It has also been shown in particular that one of these bis-benzimidazole derivatives, known as “Hoechst 332581”, for which Ar—R₁ is p-phenol, R₂ is a methyl group and R₃ is H, is also a good ligand for the minor groove of DNA, in addition to being a fluorophore. “Hoechst 33258” has thus been described as being useful for visualizing newly synthesized DNA and for determining the number of A-T base pairs present in a DNA sample (see F. G. Loontiens et al., Biochemistry, 1990, 29, pp. 9029-9039).

Many compounds similar to “Hoechst 33258” have since been synthesized. For example, an analogue for which the hydroxyl group on the terminal phenol is placed in the meta position instead of the para position has been prepared for the purpose of potentially introducing hydrogen bonding with certain functional groups of DNA (S. E. S. Ebrahimi et al., Anti-Cancer Drug Design, 1995, 10, pp. 463-479). It has been shown that this slight structural difference relative to “Hoechst 33258” does not introduce any major changes in properties, although even a very slight change in structure is liable to alter the properties of binding to DNA (see page 464 of the same document). Such derivatives are described as being useful as biological tools and as site-directed medicinal products directed towards the genome in cases of viral diseases or cancers.

The compound “Hoechst 33258” has also been modified by introducing onto the end phenol substituent various kinds of linking molecules (commonly referred to as “linkers”) such as, for example, hexakis (ethylene glycol), in order to be able to link this fluorophore covalently to oligo (deoxynucleotides), thereby making it possible to increase the stability of the hybridization complex formed and to monitor the success of this hybridization by measuring the fluorescence (K. Wiederholt et al., J. Am. Chem. Soc., 1996, 118, pp. 7055-7062; Sharanabasava B. Rajur et al., J. Org. Chem., 1997, 62, pp. 523-529).

Conjugates between “Hoechst 33258” and polyethylene glycol (“PEG”) have also been formed in order to allow the separation of DNA fragments amplified by polymerization chain reaction (“PCR”), which are identical in length but different as regards their base composition, by virtue of the binding properties of the compound “Hoechst 33258” to DNA (M. Müller et al., Nucleic Acid Research, 1997, Vol. 25, No. 24, pp. 5125-5126).

Finally, analogues of “Hoechst 33258” bearing an alkyl chain containing 5, 8 or 12 carbon atoms on the oxygen on the terminal phenol have also been synthesized. It has been shown that these analogues bind the minor groove of DNA and that this results in inhibition of the transcription of certain specific genes of cancer cells. It has also been shown that these analogues induce a selective toxicity with respect to human melanoma cells (S. S. C. Wong et al., Biochemical Pharmacology, 1994, Vol. 47, No. 5, pp. 827-837).

Moreover, it is known that cationic lipids are agents for transfecting DNA into cells. Specifically, on account of their positive overall charge, they interact spontaneously with DNA, which is negative overall, thus forming, by ionic interactions, compacted nucleolipid complexes which are capable of binding to cell membranes, and allow the intracellular release of the DNA. However, the use of these cationic lipids as transfecting agents poses many further problems, and their efficacy remains to be improved. In particular, it has been observed that, in order to obtain effective, stable nucleolipid complexes, it is generally necessary for these complexes to be highly cationic. However, it would be desirable to be able to provide noncationic or less cationic vectors so as to form with the nucleic acid particles that are neutral or negative overall, for various reasons:

on account of their overall positive charge, the complexes formed between the nucleic acid and the transfer vectors have a tendency to be captured by the reticuloendothelial system, thus limiting their removal,

on account of the overall positive charge on the complexes formed, the plasma proteins have a tendency to be adsorbed onto their surface, resulting in a loss of the transfecting power,

in a context of local injection, the presence of a large positive overall charge prevents nucleic acid complexes from diffusing beyond the site of administration, since the complexes become adsorbed onto the extracellular matrices; the complexes can thus no longer reach the target cells, which, consequently, results in a reduction in the transfer efficacy relative to the amount of complexes injected,

and, lastly, cationic lipids or polymers have an inflammatory effect, which has been observed on many occasions.

An alternative to cationic lipids for transferring nucleic acids has thus been proposed in the thesis by J. S. Rémy (Synthèse et Utilisation in vitro de nouveaux vecteurs de transfert de gènes [Synthesis and use in vitro of novel gene-transfer vectors], Jean-Serge REMY, Université Louis Pasteur de Strasbourg, viva of Apr. 13, 1994), in the context of which oligopyrroles coupled to hydrocarbon-based fatty chains were synthesized in order to form complexes with DNA, in particular by virtue of the ligand properties of peptide oligopyrroles for the minor groove of DNA. However, the oligopyrrole lipid derivatives synthesized were found to be slightly toxic and showed no transfecting efficacy. Such vectors thus did not appear to be advantageous relative to cationic lipids.

### SUMMARY OF THE INVENTION

It has now been found that the oligobenzimidazole derivatives of general formula (I):

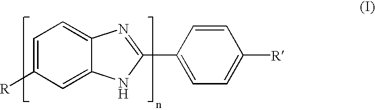

in which

R represents a hydrogen atom, a carboxyl, alkoxycarbonyl, carbamoyl or alkylcarbamoyl radical or a piperazinyl group optionally substituted in position −4 with an alkyl containing 1 to 4 straight or branched carbon atoms, or alternatively R represents an imidazolyl group,

n is an integer equal to 2, 3, 4 or 5, and

R′ represents a group —O—R₃, —S—R₃, NHR₃ or —O—CO—NH—R₃ and R₃ represents an alkyl group,

or alternatively R′ represents a group —NR₄R₅ or —O—CO—NR₄R₅ and R₄ and R₅, which may be identical or different, each represent an alkyl group,

the alkyl radicals mentioned above being, except where specified otherwise, straight or branched, optionally saturated and containing 12 to 22 carbon atoms, as well as the salts thereof, show DNA binding properties and fluorescence properties that are particularly advantageous in the context of an in vitro, in vivo or ex vivo administration of DNA and its visualization.

Specifically, the compounds of general formula (I) according to the present invention constitute derivatives of “Hoechst 33258” coupled to one or more hydrocarbon-based fatty chains, and it has been shown that these compounds are DNA ligands, and in particular for the minor groove of DNA, but that, unlike cationic lipids, they do not compact said DNA. More specifically, the oligobenzimidazole derivatives of general formula (I) according to the invention form nonionic hydrogen bonds with DNA. They thus make it possible to stabilize DNA in a context of DNA production and/or purification. In addition, it has also been shown that the oligobenzimidazole derivatives according to the invention conserve the same fluorescence properties as “Hoechst 33258” when they are combined with DNA, thus allowing the DNA to be visualized. Finally, it has been demonstrated that, unlike lipidic oligopyrroles, the oligobenzimidazole derivatives according to the present invention allow the transfer of DNA into cells while at the same time protecting this DNA against the degradation caused by endonucleases.

According to one variant of the invention, the oligobenzimidazole derivatives have the general formula (II):

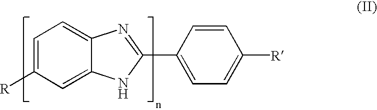

in which

R represents a hydrogen atom or a piperazinyl group optionally substituted in position −4 with an alkyl containing 1 to 4 straight or branched carbon atoms,

n is an integer equal to 2 or 3, and

R′ represents a group —OR₃, NHR₃ or —O—CO—NH—R₃ and R₃ represents an alkyl group,

or alternatively R′ represents a group NR₄R₅ or —O—CO—NR₄R₅ and R₄ and R₅, which may be identical or different, each represent an alkyl group,

the alkyl radicals mentioned above being, except where otherwise specified, straight or branched, optionally saturated and containing 12 to 22 carbon atoms, it being understood that R′ is other than OR₃ with R₃ representing a dodecyl substituent when R represents 4-methylpiperazinyl and that n is equal to 2, as well as the salts thereof.

For the purposes of the invention, the straight or branched, optionally saturated alkyl substituents containing 12 to 22 carbon atoms are also referred to as “fatty chains”. The fattychain (s) can in particular contain 12, 14, 16 or 18 carbon atoms. They may be in particular (CH₂)₁₁CH₃, (CH₂)₁₃CH₃, (CH₂)₁₅CH₃ or (CH₂)₁₇CH₃ fatty chains.

Besides the provisions hereinabove, the present invention also comprises other characteristics and advantages which will emerge from the examples and figures which follow, and which should be considered as illustrating the invention without limiting its scope. In particular, the Applicant proposes, in a nonlimiting manner, various operating protocols as well as reaction intermediates which can be used to prepare the transfer agents of general formula (I). Needless to say, it is within the capabilities of a person skilled in the art to be inspired by these protocols or intermediate products to develop similar processes in order to lead to these same compounds.

### BRIEF DESCRIPTION OF THE FIGURES

FIG. 1: Structure of the oligobenzimidazole derivatives (1) and (2) whose preparation is outlined in Examples 1 and 2.

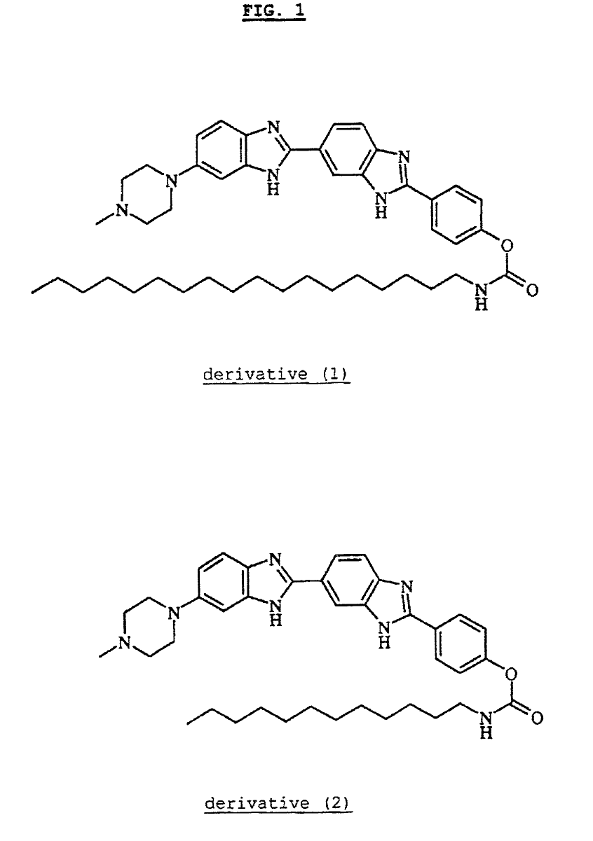

FIG. 2: Fluorescence emission signal at 450 nm of DNA/derivative (1) complexes (solid-line curve) and of derivative (1) alone (dotted-line curve) as a function of increasing amounts of derivative (1) in nmol/μg of DNA. The DNA concentration is 50 μg/ml.

FIG. 3: Fluorescence emission signal at 450 nm of DNA/derivative (2) complexes (solid-line curve) and of derivative (2) alone (dotted-line curve) as a function of increasing amounts of derivative (2) in nmol/μg of DNA. The DNA concentration is 50 μg/ml.

FIG. 4: Agarose gel of a DNA plasmid complexed with the derivatives (1) and (2), at various derivative concentrations expressed in nmol of product per μg of DNA.

“*”: yellow band characteristic of the derivative not complexed to DNA, and which thus remains at the point of injection.

“**”: blue band characteristic of the product complexed to DNA.

FIG. 5: Agarose gel of a DNA plasmid complexed with the derivatives (1) and (2), at various derivative concentrations expressed in nmol of product per μg of DNA. The gel was revealed under the same UV lamp as for FIG. 4, but with ethidium bromide.

FIG. 6: Agarose gel (0.8%) of 1 μg of a DNA plasmid associated with increasing amounts of a cationic lipid of formula:

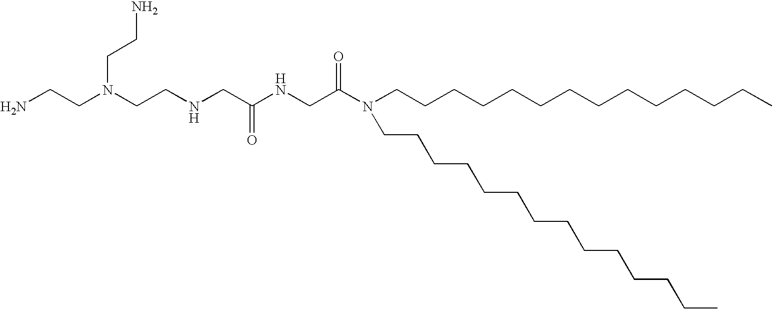

as described in patent application WO 97/18185, the amounts being expressed in nmol of cationic lipid per μg of DNA. The bands are revealed with ethidium bromide and by absorption under a UV lamp.

FIG. 7: Schematic representation of the plasmid pXL3031 used in the experiments of DNA transfer into cells.

### DETAILED DESCRIPTION OF THE INVENTION

Various publications, patents and patent applications are cited herein, the disclosures of which are hereby incorporated by reference in their entireties

The oligobenzimidazole derivatives of general formula (I) can be obtained according to methods analogous to those described in patent FR 1 519 964. This is more particularly the case when it is desired to obtain derivatives for which R represents an optionally substituted piperazinyl substituent. Specifically, it is possible in this case to start with the commercial product “Hoechst 33258” and to graft the substituent R′ as defined in the general formula (I) onto the hydroxyl group of the terminal phenol according to conventional methods known to those skilled in the art or according to similar methods. Moreover, the oligobenzimidazole derivatives of general formula (I) can also be obtained either by solid phase synthesis or by liquid phase synthesis.

A—Preparation in Liquid Phase

It is possible, in a nonlimiting manner, to perform the process in the following way:

1) 3,4-Dinitrobenzaldehyde is coupled with commercial 1,2-diaminobenzene so as to obtain, after spontaneous cyclization, a nitro derivative of general formula (III):

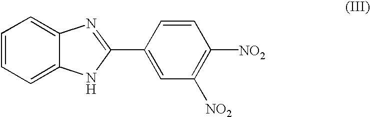

The coupling is carried out in dioxane in the presence of diiodine. The process is preferably performed at a temperature of between 10° C. and 40° C. for about 24 hours.

The 3,4-dinitrobenzaldehyde can be obtained in the following way:

a) Commercial 3,4-dinitrobenzoic acid is converted into the corresponding acyl halide according to the conventional methods, known to those skilled in the art, for obtaining an acyl halide from an acid or according to similar methods. For example, the process is performed in the presence of a reagent such as thionyl chloride, phosphorus trichloride or tribromide, or phosphorus pentachloride or pentabromide, at a temperature of between about 70° C. and 90° C. According to another method, the process is performed in the presence of triphenylphosphine in tetrachloromethane.

b) The 3,4-dinitrobenzylcarbonyl halide is then reduced to the corresponding alcohol according to the conventional methods, known to those skilled in the art, for obtaining an alcohol from an acyl halide or according to similar methods. For example, the process can be performed in the presence of lithium borohydride in a suitable solvent (for example tetrahydrofuran) at very low temperature, for example at −78° C.

c) The alcohol obtained in the preceding step is finally oxidized to 3,4-dinitrobenzaldehyde according to the conventional methods, known to those skilled in the art, for obtaining an aldehyde from an alcohol or according to similar methods. For example, chromium oxide can be used as oxidizing agent and the process can be performed in the presence of trimethylsilyl chloride. In this case, the temperature used is between about 10° C. and 40° C. in a suitable organic solvent such as, for example, dimethylformamide, chlorinated solvents, etc.

2) The nitro derivative of general formula (III) is then reduced so as to obtain a diamino derivative of general formula (IV):

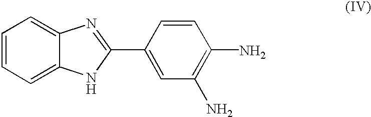

The reduction is carried out according to the conventional methods, for example by catalytic hydrogenation in acidic medium in the presence of Raney nickel or palladium-on-charcoal, in an alcohol and at a temperature of between 20 and 60° C. Methanol or ethanol can be used as alcohol. Another alternative consists in performing the process by the action of stannous chloride in acidic aqueous medium at a temperature of between 20 and 100° C., or alternatively by reduction with iron in acidic aqueous and alcoholic medium at a temperature of between 20 and 100° C. The acidic aqueous solution can be, for example, an aqueous hydrochloric acid solution. The alcoholic solution can be, for example, methanol or ethanol.

3) The coupling and reduction steps as described in 1) and 2) are repeated n−2 times successively so as to give a diamino derivative of general formula (V):

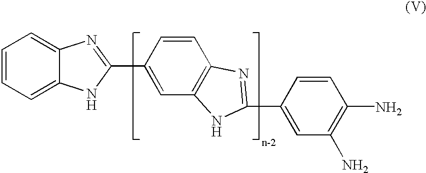

in which n represents an integer chosen from 2, 3, 4 and 5.

4) The diamino derivative of general formula (V) obtained previously is then coupled with a nitrobenzaldehyde derivative of general formula (VI):

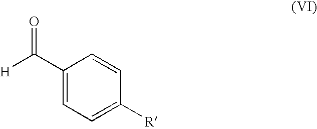

in which R′ is as defined in the general formula (I), so as to give the derivative of general formula (VII):

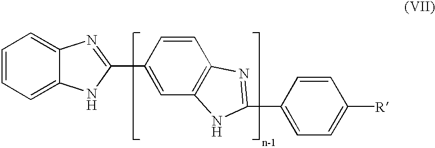

in which R′ is as defined above.

Preferably, the coupling is carried out in dioxane in the presence of diiodine. The process is preferably performed at a temperature of between 10° C. and 40° C. for about 24 hours. According to another method, the process is performed in the presence of dichlorodicyanoquinone (DDQ) in a suitable solvent, for example N,N-dimethylformamide, N-methylpyrrolidinone, dimethylacetamide, acetonitrile, dichloromethane, toluene, benzene, etc.

The benzaldehyde derivative of general formula (VI) is either commercial or is obtained:

a) by alkylation of commercial 4-hydroxybenzaldehyde according to the conventional methods known to those skilled in the art or according to similar methods when R′ represents a group OR₃,

b) by reduction followed by an alkylation of the commercial 4-nitrobenzaldehyde according to the conventional methods known to those skilled in the art or according to similar methods when R′ represents a group NHR₃, or alternatively

c) by nucleophilic addition of the acid derivative COOH—NHR₃ or COOH—NR₄R₅ onto the commercial 4-hydroxy-benzaldehyde according to the conventional methods known to those skilled in the art or according to similar methods when R′ represents a group —O—CO—NHR₃ or —O—CO—NR₄R₅.

5) When it is desired for the derivatives of general formula (I) according to the present invention to bear a substituent R representing an optionally substituted piperazinyl group or an imidazolyl group, then the first step of the process described above is carried out starting with 1,2-diaminobenzene substituted in position −4 with the group R.

B—Preparation in Solid Phase, First Variant

The oligobenzimidazole derivatives of general formula (I) according to the present invention can also be prepared in solid phase. This is more particularly the case when it is desired for the derivatives of general formula (I) according to the present invention to bear a substituent R representing a carboxyl, alkoxycarbonyl, carbamoyl or alkylcarbamoyl group. In this case, the process may be performed as follows:

1) Commercial 3,4-diaminobenzoic acid is grafted onto a conventional Wang-type resin substituted with a bromine or iodine atom or with a hydroxyl group, or any other suitable resin, so as to obtain the substituted resin of general formula (VIII):

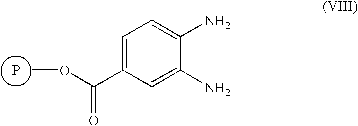

When the starting resin is substituted with a halogen atom, the coupling is carried out in the presence of a cesium salt and a non-nucleophilic base in N-ethyldiisopropylamine, in a suitable aprotic solvent. Non-nucleophilic bases which may be used, for example, are tertiary amines, calcium carbonate or sodium bicarbonate. Even more preferably, the bases used are tertiary amines, for example triethylamine (TEA) or N-ethyldiisopropylamine (DIEA). The suitable solvents can be chosen from N-methylpyrrolidinone and dimethylformamide.

2) 3,4-Dinitrobenzaldehyde is coupled with the substituted resin of general formula (VIII) obtained in the preceding step, so as to give, after spontaneous cyclization, a nitro derivative of general formula (IX):

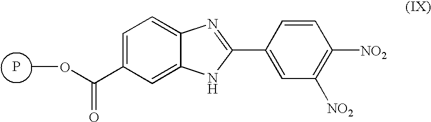

The coupling is carried out in dioxane in the presence of diiodine. The process is preferably performed at a temperature of between 10° C. and 40° C. for about 24 hours. The 3,4-dinitrobenzaldehyde is obtained in the same way as described above for the preparation in liquid phase.

3) The nitro derivative of general formula (IX) obtained is then reduced so as to give a diamino derivative of general formula (X):

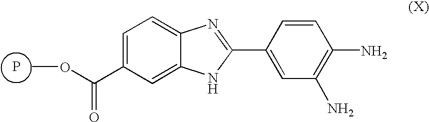

The reduction is preferably carried out in the presence of a Lewis acid in a suitable solvent. Lewis acids which are used, for example, are tin chloride or chromium chloride. Suitable solvents which are used, for example, are N,N-dimethylformamide or N-methylpyrrolidinone.

4) The coupling and reduction steps as described above in 2) and 3) are repeated a further n−2 times successively so as to give a diamino derivative of general formula (XI):

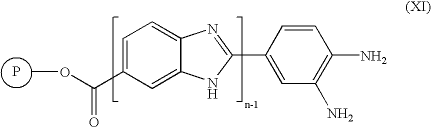

in which n represents an integer chosen from 2, 3, 4 and 5.

5) The diamino derivative of general formula (XI) obtained above is then coupled with a nitrobenzaldehyde derivative of general formula (VI):

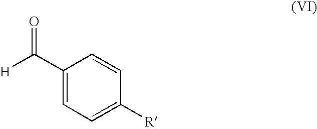

in which R′ is as defined in the general formula (I), so as to give a derivative of general formula (XII):

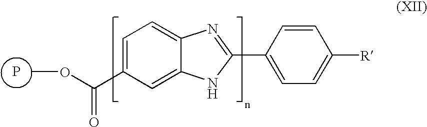

in which R′ is as defined above.

Preferably, the coupling is carried out in dioxane in the presence of diiodine. The process is preferably performed at a temperature of between 10° C. and 40° C. for about 24 hours. According to another method, the process is performed in the presence of dichlorodicyanoquinone (DDQ) in a suitable solvent chosen from N,N-dimethylformamide and N-methyl-pyrrolidinone.

The benzaldehyde derivative of general formula (VI) is either commercial or it is obtained as indicated above for the preparation in liquid phase.

6) The derivative obtained in the preceding step is then cleaved from the resin, thus giving the acid of general formula (XIII):

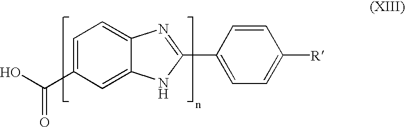

in which R′ and n are as defined above.

The cleavage of the resin is carried out according to the conventional methods known to those skilled in the art or according to any other similar method. For example, the process is performed in the presence of trifluoroacetic acid at a temperature of between 10° C. and 50° C.

7) In order to obtain the oligobenzimidazole derivatives of general formula (I), the process is performed in the following way, depending on the meaning of R:

a) when R represents an alkoxycarbonyl radical, the process is performed according to the conventional esterification methods, known to those skilled in the art, which do not adversely affect the rest of the molecule, in particular by application or adaptation of the methods described in Tetrahedron, 33, 683 (1977), Tetrahedron Letters, 4475 (1978) or Bull. Soc. Chim. Japan, 40, 2380 (1967),

b) when R represents a carbamoyl or alkylcarbamoyl radical, the process is performed according to the conventional methods for converting acids into amides, known to those skilled in the art and which do not adversely affect the rest of the molecule, for example by treatment with ammonia or with a suitable primary amine (for R representing an alkylcarbamoyl radical).

C—Preparation in Solid Phase, Second Variant

According to another variant, the synthesis in solid phase can be carried out as follows:

1) Commercial 3-nitro-4-aminobenzoic acid is grafted onto a conventional Wang-type resin substituted with a bromine or iodine atom or with a hydroxyl group, or any other suitable similar resin, so as to give the substituted resin of general formula (XIV):

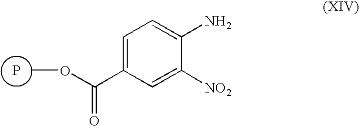

When the starting resin is substituted with a halogen atom, the coupling is carried out in the presence of a cesium salt and a non-nucleophilic base in N-ethyldiisopropylamine, in a suitable aprotic solvent. Non-nucleophilic bases which can be used, for example, are tertiary amines, calcium carbonate or sodium bicarbonate. Even more preferably, the bases used are tertiary amines, for example triethylamine (TEA) or N-ethyldiisopropylamine (DIEA). The suitable solvents can be chosen from N-methylpyrrolidinone and dimethylformamide.

2) 4-Fluoro-3-nitrobenzylcarbonyl chloride is added to the substituted resin of general formula (XIV) obtained in the preceding step, thus giving the substituted resin of general formula (XV) below:

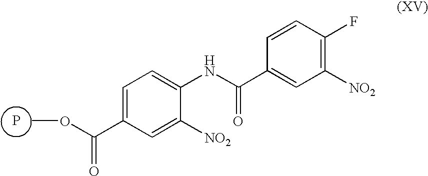

The coupling is carried out according to the conventional peptide coupling methods (Bodanski M., Principles and Practices of Peptide Synthesis, Ed. Springer-Verlag) or by any similar method known to those skilled in the art. In particular, the reaction is generally carried out in the presence of a non-nucleophilic base in suitable aprotic solvents, at a temperature of between 0 and 100° C., the pH being adjusted to between 9 and 11.

By way of example, chloroform, dimethylformamide, methylpyrrolidone, acetonitrile, dichloromethane, toluene or benzene can be used as solvent.

The non-nucleophilic bases employed are preferably tertiary amines, calcium carbonate or sodium bicarbonate. Even more preferably, the bases used are tertiary amines such as, for example, triethylamine (TEA) or N-ethyldiisopropylamine.

Advantageously, the peptide coupling is carried out at between 0 and 50° C. and preferably between 10 and 30° C.

The 4-fluoro-3-nitrobenzylcarbonyl chloride is obtained from the corresponding commercial acid according to any method known to those skilled in the art for obtaining an acyl halide from an acid. For example, the process can be performed by the action of thionyl chloride at a temperature of between about 70° C. and 90° C.

3) Next, the fluorine atom on the substituted resin of general formula (XV) obtained in the preceding step is converted into an amine function, so as to give a substituted resin of general formula (XVI):

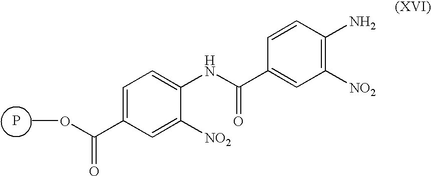

The amination is performed according to the conventional methods known to those skilled in the art for converting a halogen atom into an amino function, for example by nucleophilic substitution working in the presence of ammonia in a suitable solvent, for example N,N-dimethylformamide.

4) Steps 2) and 3) as described above are repeated a further n−2 times successively so as to give a substituted resin of general formula (XVII):

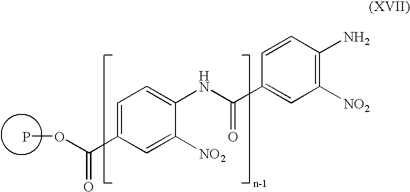

5) The substituted resin of general formula (XVII) obtained above is then coupled with an acyl halide derivative of general formula (XVIII):

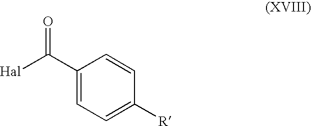

in which Hal represents a halogen atom chosen from chlorine, bromine, iodine and fluorine, and R′ is as defined above,

so as to give a substituted resin of general formula (XIX):

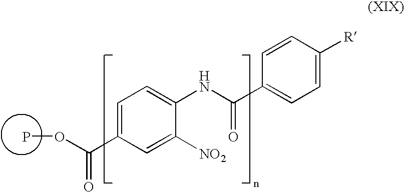

The coupling is carried out according to the conventional peptide coupling methods (Bodanski M., Principles and Practices of Peptide Synthesis, Ed. Springer-Verlag) or by any similar method known to those skilled in the art. In particular, the reaction is generally carried out in the presence of a non-nucleophilic base in suitable aprotic solvents, at a temperature of between 0 and 100° C., the pH being adjusted to between 9 and 11.

By way of example, chloroform, dimethylformamide, methylpyrrolidone, acetonitrile, dichloromethane, toluene or benzene can be used as solvent.

The non-nucleophilic bases employed are preferably tertiary amines, calcium carbonate or sodium bicarbonate. Even more preferably, the bases used are tertiary amines such as, for example, triethylamine (TEA) or N-ethyldiisopropylamine.

Advantageously, the peptide coupling is carried out at between 0 and 50° C. and preferably between 10 and 30° C.

The acyl halide derivative of general formula (XVIII) is either Commercial or is obtained from the corresponding acid according to any method known to those skilled in the art for obtaining an acyl halide from an acid. For example, the process can be performed by the action of thionyl chloride at a temperature of between about 70° C. and 90° C.

The corresponding acid derivative is either commercial or is obtained:

a) by alkylation of commercial 4-hydroxybenzoic acid according to the conventional methods known to those skilled in the art or according to similar methods when R′ represents a group OR₃,

b) by reduction followed by an alkylation of commercial 4-nitrobenzoic acid according to the conventional methods known to those skilled in the art or according to similar methods when R′ represents a group NHR₃, or alternatively

c) by nucleophilic addition of the acid derivative COOH—NHR₃ or COOH—NR₄R₅ on commercial 4-hydroxybenzoic acid according to the conventional methods known to those skilled in the art or according to similar methods when R′ represents a group —O—CO—NHR₃ or —O—CO—NR₄R₅.

6) The substituted resin of general formula (XIX) obtained in the preceding step is then reduced so as to give a resin substituted with a polycyclized product of general formula (XX):

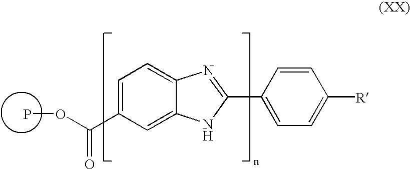

The reduction is preferably carried out in the presence of a Lewis acid in a suitable solvent. Lewis acids which are used, for example, are tin chloride or chromium chloride. Suitable solvents which are used, for example, are N,N-dimethylformamide or N-methylpyrrolidinone.

7) The polycyclized product obtained in the preceding step is cleaved from the resin, thus giving a derivative of general formula (XXI):

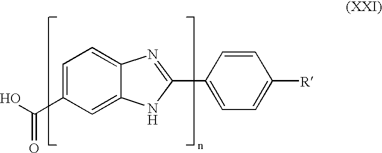

The cleavage from the resin is carried out according to the conventional methods known to those skilled in the art or according to any other similar method. For example, the process is performed in the presence of trifluoroacetic acid at a temperature of between 10° C. and 50° C.

8) Finally, the oligobenzimidazole derivatives of general formula (I) according to the invention are obtained from the derivative of general formula (XXI) obtained in the preceding step, by substitution of the acid function with the group R, R being defined as above, in a manner analogous to the methods described above in 7) for the first preparation variant in solid phase.

The novel oligobenzimidazole derivatives according to the present invention, as well as the synthetic intermediates thereof, can optionally be purified by physical methods such as crystallization or chromatography.

Moreover, the oligobenzimidazole lipidic derivatives according to the invention, as well as the intermediates thereof, can be converted into metal salts or into addition salts with nitrogenous bases according to methods that are known per se. These salts can be obtained according to the usual methods which do not adversely affect the rest of the molecule, in particular by the action of a metal base (for example an alkali or alkaline-earth metal base), ammonia or an amine on a product mentioned above in a suitable solvent such as an alcohol, an ether or water, or by exchange reaction with an organic acid salt. The salt formed precipitates after optional concentration of its solution, and is separated by filtration, decantation and/or lyophilization.

The oligobenzimidazole lipidic derivatives according to the invention can also be converted into addition salts with acids. The compounds of general formula (I) obtained in the form of these salts can be released and converted into salts of other acids according to the usual methods.

Examples of pharmaceutically acceptable salts which may be mentioned are the salts with alkali metals (sodium, potassium or lithium) or with alkaline-earth metals (magnesium or calcium), the ammonium salt, the salts of nitrogenous bases (ethanolamine, diethanolamine, trimethylamine, triethylamine, methylamine, propylamine, diisopropylamine, N,N-dimethylethanolamine, benzylamine, dicyclohexylamine, N-benzylphenethylamine, N,N′-dibenzylethylenediamine, diphenylenediamine, benzhydrylamine, quinine, choline, arginine, lysine, leucine, dibenzylamine), as well as the addition salts with inorganic acids (hydrochlorides, hydrobromides, sulfates, nitrates or phosphates) or organic acids (succinates, fumarates, maleates, methanesulfonates, p-toluenesulfonates or isethionates).

Another subject of the invention relates to compositions comprising an oligobenzimidazole derivative as defined above and a nucleic acid.

Another subject of the invention relates to the compositions as defined above and also comprising one or more adjuvants.

Adjuvants which may be mentioned, for example, are neutral colipids which are capable of combining with the complexes formed between DNA and the oligobenzimidazole derivatives according to the invention and of improving the transfecting power thereof. In particular, natural or synthetic lipids which are zwitterionic or devoid of ionic charges under physiological conditions can be used. Representative examples of neutral colipids include cholesterol, dioleylphosphatidylethanolamine (DOPE), oleoylpalmitoylphosphatidylethanolamine (POPE), distearoyl-, dipalmitoyl- and dimyristoylphosphatidyl-ethanolamine as well as the derivatives thereof N-methylated 1 to 3 times, phosphatidyl glycerols, diacyl glycerols, glycosyldiacyl glycerols, cerebrosides (in particular such as galacto-cerebrosides), sphingolipids (in particular such as sphingomyelins) or asialogangliosides (in particular such as asialoGM1 and GM2).

These various neutral colipids can be obtained either by synthesis or by extraction from organs (for example such as the brain) or from eggs, by conventional techniques known to those skilled in the art. For example, the extraction of natural lipids can be carried out using organic solvents (see also Biochemistry, Lehninger).

The compositions according to the invention generally comprise 0.01 to 20 [lacuna] of a neutral colipid per one equivalent of nucleic acid (in mol/mol) and preferably 0.05 to 5 equivalents of a neutral colipid.

Adjuvants which can also be used are compounds which improve the bioavailability, for example polyethylene glycol.

According to another embodiment, the compositions of the present invention can also contain a targeting element for orientating the transfer of the nucleic acid. This targeting element can be an extracellular targeting element for orienting the transfer of DNA toward certain desired cell types or certain desired tissues (tumor cells, liver cells, hematopoietic cells, etc.). It can also be an intracellular targeting element for orienting the transfer of nucleic acid toward certain preferred cell compartments (mitochondria, nucleus, etc.).

Among the targeting elements which can be used in the context of the invention, mention may be made of sugars, peptides, proteins, oligonucleotides, lipids, neuromediators, hormones, vitamins or derivatives thereof. Preferably, they are sugars, peptides or proteins such as antibodies or antibody fragments, cell receptor ligands or fragments thereof, receptors or receptor fragments, etc. In particular, they may consist of ligands of growth factor receptors, of cytokine receptors, of receptors of cell lectin type, or ligands with an RGD sequence which have an affinity for the receptors for adhesion proteins such as integrins. Mention may also be made of the receptors for transferin, for HDLs and LDLs, or the folate transporter. The targeting element can also be a sugar for targeting lectins, such as the receptors for asialoglycoproteins or for sialyls such as sialyl Lewis X, or alternatively an antibody Fab fragment, or a single-chain antibody (ScFv).

The respective amounts of each component can be easily adjusted by a person skilled in the art as a function of the oligobenzimidazole derivative used, the nucleic acid or the adjuvant(s) and the desired applications (in particular the type of cells to be transfected).

For the purposes of the invention, the term “nucleic acid” means double-stranded deoxyribonucleic acids forming a double helix which comprises a minor groove and a major groove. These may be natural or artificial sequences, and in particular genomic DNA (gDNA), complementary DNA (cDNA), hybrid sequences or synthetic or semisynthetic sequences. These nucleic acids can be of human, animal, plant, bacterial, viral, etc. origin. They can be obtained by any technique known to those skilled in the art, and in particular by screening libraries, by chemical synthesis or by mixed methods including chemical or enzymatic modification of sequences obtained by screening libraries. They can be chemically modified.

According to one specific embodiment, the nucleic acids consist of vectors, in particular expression vectors, recombinant vectors, plasmids, episomes, etc. The said vectors comprise a coding sequence and all the elements necessary for expressing said coding sequence, in particular elements for regulating the expression of the nucleic acid to be inserted, such as promoters and activating sequences (“enhancers”) or suitable sequences for starting and stopping transcription, as well as other elements such as, for example, sequences encoding a functional or nonfunctional replication origin, marker genes, regions for binding to other cell components, signal sequences, polyadenylation sequences, etc.

The expression “coding sequence” means a gene of therapeutic interest placed in phase with regulation sequences, for example one or more promoters and a transcription terminator, which are active in the target cells.

For the purposes of the invention, the expression “gene of therapeutic interest” means in particular any gene encoding a protein product which has a therapeutic effect. The protein product thus encoded can be, in particular, a protein or a peptide. This protein product can be an exogenous homolog or endogenous with respect to the target cell, i.e. a product which is normally expressed in the target cell when this cell exhibits no pathology. In this case, the expression of a protein makes it possible, for example, to overcome an insufficient expression in the cell or the expression of a protein which is inactive or weakly active on account of a modification, or alternatively to overexpress said protein. The gene of therapeutic interest can also encode a mutant of a cell protein, which has increased stability, modified activity, etc. The protein product can also be heterologous with respect to the target cell. In this case, a protein expressed can, for example, complement or provide an activity which is deficient in the cell, thus allowing it to control a pathology, or to stimulate an immune response.

Among the therapeutic products which may be mentioned more particularly, for the purposes of the present invention, are enzymes, blood derivatives, hormones, lymphokines: interleukins, interferons, TNF, etc. (FR 92/03120), growth factors, neurotransmitters or the synthetic enzymes or precursors thereof, trophic factors (BDNF, CNTF, NGF, IGF, GMF, aFGF, bFGF, NT3, NT5, HARP/pleiotrophin, etc.), apolipoproteins (ApoAI, ApoAIV, ApoE, etc., FR 93/05125), dystrophin or a minidystrophin (FR 91/11947), CFTR protein associated with mucoviscidosis, tumor suppressant genes (p53, Rb, Rap1A, DCC, k-rec, etc., FR 93/04745), genes encoding factors involved in clotting (factors VII, VIII and IX), genes involved in DNA repair, suicide genes (thymidine kinase, cytosine deaminase), the genes for hemoglobin or for other transport proteins, metabolic and catabolic enzymes, etc.

The nucleic acid of therapeutic interest can also be an antisense sequence or gene, whose expression in the target cell makes it possible to control cellular mRNA transcription or gene expression. Such sequences can, for example, be transcripted in the target cell into RNA complementary to cellular mRNA and thus block its translation into protein, according to the technique described in patent EP 140 308. The therapeutic genes also comprise the sequences encoding ribozymes, which are capable of selectively destroying target RNAs (EP 321 201).

As mentioned above, the nucleic acid can also comprise one or more genes encoding an antigenic peptide capable of generating an immune response in man or animals. In this specific embodiment, the invention allows the preparation either of vaccines or of immunotherapeutic treatments applied to man or animals, in particular against microorganisms, viruses or cancers. They may be, in particular, antigenic peptides specific for the Epstein Barr virus, the HIV virus, the hepatitis B virus (EP 185 573), the pseudorabies virus, the “syncitia forming virus”, other viruses or alternatively antigenic peptides specific for tumors (EP 259 212).

Preferably, the nucleic acid also comprises sequences allowing the expression of the gene of therapeutic interest and/or the gene encoding the antigenic peptide in the desired cell or organ. These may be sequences which are naturally responsible for the expression of the gene under consideration when these sequences are capable of functioning in the infected cell. They may also be sequences of different origin (responsible for the expression of other proteins, or even synthetic sequences). In particular, they may be promoter sequences of eukaryotic or viral genes. For example, they may be promoter sequences derived from the genome of the cell which it is desired to infect. Similarly, they may be promoter sequences derived from the genome of a virus. In this respect, mention may be made, for example, of the E1A, MLP, CMV, RSV, etc. gene promoters. In addition, these expression sequences can be modified by addition of activation sequences, regulation sequences, etc. It may also concern an inducible or repressible promoter.

Moreover, the nucleic acid can also comprise, in particular upstream of the gene of therapeutic interest, a signal sequence directing the therapeutic product synthesized into the secretion pathways of the target cell. This signal sequence can be the natural signal sequence of the therapeutic product, but it can also be any other functional signal sequence, or an artificial signal sequence. The nucleic acid can also comprise a signal sequence directing the therapeutic product synthesized toward a specific cell compartment.

The compositions according to the invention can be formulated for the purpose of topical, cutaneous, oral, rectal, vaginal, parenteral, intranasal, intravenous, intramuscular, subcutaneous, intraocular, transdermal, intratracheal, intraperitoneal, etc. administration. Preferably, the compositions of the invention contain a vehicle which is pharmaceutically acceptable for an injectable formulation, in particular for a direct injection into the desired organ, for topical administration (onto skin and/or mucous membranes) or for administration by aerosolization. These compositions may be, in particular, sterile isotonic solutions or dry compositions, in particular lyophilized compositions, which, on addition of sterilized water or physiological saline, depending on the case, allow the constitution of injectable solutions. The doses of nucleic acids used for the injection and the number of administrations can be adapted as a function of various parameters, and in particular as a function of the method of administration used, the pathology concerned, the gene to be expressed or the desired duration of the treatment. As more particularly regards the method of administration, this may be either a direct injection into the tissues, for example into tumors, or into the circulatory pathways, or a treatment of cells in culture followed by reimplanting them in vivo, by injection or grafting. The tissues concerned in the context of the present invention are, for example, the muscles, the skin, the brain, the lungs, the liver, the spleen, bone marrow, the thymus, the heart, the lymph, the blood, the bones, the cartilages, the pancreas, the kidneys, the bladder, the stomach, the intestines, the testicles, the ovaries, the rectum, the nervous system, the eyes, the glands, the connective tissues, etc.

A subject of the invention is also the use of the oligobenzimidazole derivatives as defined above for the transfer of nucleic acids into cells in vitro, in vivo or ex vivo. More specifically, a subject of the present invention is the use of the compounds as defined above to prepare a medicinal product for transferring nucleic acid into cells. The nucleic acid contained in said medicinal product encodes a protein product or nucleic acid product, or constitutes said nucleic acid product, which is capable of correcting diseases in vivo or ex vivo in which said protein product or nucleic acid product is involved.

The invention also relates to a method for transferring nucleic acids into cells, comprising a first step during which the nucleic acid is placed in contact with at least one oligobenzimidazole derivative according to the invention and optionally with one or more adjuvants and/or one or more physiologically compatible vehicles to form a complex, and a second step which consists in placing the complex thus formed in contact with cells.

The placing in contact of cells with the complex can be carried out by incubating the cells with said complex (for in vitro or ex vivo uses), or by injecting or aerosolizing the complex in an organism (for in vivo uses). The incubation is preferably carried out in the presence of, for example, from 0.01 to 1000 μg of nucleic acid per 10⁶ cells. For an in vivo administration, nucleic acid doses ranging from 10⁻⁴ to 10 mg can be used, for example.

The oligobenzimidazole derivatives according to the invention can be used to transfer nucleic acids into primary cells or into established lines. These can be fibroblast cells, muscle cells, nerve cells (neurons, astrocytes, glial cells), liver cells, hematopoietic cells (lymphocytes, CD34, dendritic cells, etc.), epithelial cells, etc. in differentiated or pluripotent (precursor) form.

Finally, the uses of the compositions according to the invention may concern both man and any animal such as sheep, cattle, pets (dogs, cats, etc.), horses, fish, etc.

### EXAMPLES

Dodecyl isocyanate, octadecyl isocyanate, N-ethyldiisopropylamine, “Hoechst 33258”, Wang-bromopolystyrene resin, iodine, cesium iodide, 3,4-diaminobenzoic acid, 1,2-dianiline, 3,4-dinitrobenzoic acid, thionyl chloride, pyridine, chromium oxide, trimethylsilyl chloride, lithium borohydride and stannous chloride are all commercially available products.

The proton NMR (nuclear magnetic resonance) spectra were recorded on Brucker 250 and 400 MHz spectrometers.

The HPLC (high performance liquid chromatography) analyses were carried out on a Hitachi machine equipped with an AS-2000A autosampler, an L-6200A pump, a UV L 4000 detector at 220 nm, and a D 2500 integrator calculator. The column used to analyze the products with lipid chains, sold by Applied Biosystems, is a stainless steel column of length 3 cm and diameter 4.6 mm. The mobile phases are water and acetonitrile containing trifluoroacetic acid, and the stationary phase is Aquapore butyl 7 micron. The flow rate ranges between 1 and 4 ml/minute. The other column used to analyze the products without lipid chains, sold by Merck, is a stainless steel column of length 25 cm and diameter 4.6 mm. The mobile phases are water and acetonitrile containing trifluoroacetic acid, and the stationary phase is Lichrospher RP-18 5 micron. The flow rate is 1 ml/minute.

The thin layer chromatographies (TLCS) were carried out on 20×20 [lacuna] aluminum plates coated with silica gel.

As regards the preparative HPLC purifications, the apparatus used is an assembly for liquid phase chromatography in gradient mode, allowing UV detection. This preparative chain is composed of the following elements:

Pump A: Gilson model 305 equipped with a 50 SC head.

Pump A: Gilson model 303 equipped with a 50 SC head.

Injection pump: Gilson model 303 equipped with a 25 SC head.

Pressure unit: Gilson model 806.

Mixer: Gilson model 811 C equipped with a 23 ml head.

UV detector: Gilson model 119 equipped with a preparative cell, and set at 220 nm.

Fraction collector: Gilson model 202 equipped with carrier No. 21.

Integrator: Shimadzu model C-R6A.

Columns: Stainless steel C4 column (10μ) of length 25 cm and diameter 2.2 cm, sold by Vydac, model 214 TP 1022. Stainless steel C18 column (10μ) of length 25 cm and diameter 2.2 cm, sold by Vydac, model 218 TP 1022.

The solution of product to be purified is loaded onto the column by means of the injection pump at a flow rate of 15 or 12 ml/minute. The mobile phases are water and acetonitrile.

### Example 1

### Synthesis of Derivative (1): 4-[6-(4-methyl-1-piperazinyl)-1H,3′H-[2,5′]bisbenzimidazol-2′-yl]-1-octadecylcarbamoyloxy phenyl

0.32 mmol of “Hoechst 33258” is dissolved in 10 cm³ of dimethylformamide. 2 mmol of N-ethyl-diisopropylamine are added to this solution, followed by 1 mmol of octadecyl isocyanate. The mixture is stirred for 24 hours at 50° C. and the reaction is monitored by HPLC. The urea obtained is filtered off, under cold conditions, in the form of a precipitate due to the excess isocyanate introduced. Acetic acid is then added to pH 4 and the solvent is evaporated off.

The crude product obtained is purified by preparative HPLC. The fractions of interest are combined and lyophilized.

0.125 mmol of salified product is obtained, i.e. a yield of 39.2%.

HPLC: Rt=9.81 min.

¹H NMR spectrum (400 MHz, (CD₃)₂SO-d₆, δ in ppm: 0.86 (t, J=7 Hz: 3H); from 1.15 to 1.40 (mt: 30H); 1.51 (mt: 2H); 2.92 (s: 3H); from 3.00 to 3.15 (mt: 2H); 3.10 (mt: 2H); 3.26 (mt: 2H); 3.61 (broad d, J=10 Hz: 2H); 3.91 (broad d, J=10 Hz: 2H); 7.20 (broad s: 1H); 7.25 (broad d, J=9 Hz: 1H); 7.36 (d, J=9 Hz: 2H); 7.68 (d, J=9 Hz: 1H); from 7.80 to 7.90 (mt: 2H); 8.06 (dd, J=9 and 1.5 Hz: 1H); 8.25 (d, J=9 Hz: 2H); 8.45 (broad s: 1H); from 9.70 to 9.90 (unres. mult. 1H).

### Example 2

### Synthesis of Derivative (2): 4-[6-(4-methyl-1-piperazinyl)-1H,3′H-[2,5′]bisbenzimidazol-2′-yl]-1-dodecylcarbamoyloxy phenyl

0.32 mmol of “Hoechst 33258” is dissolved in 10 cm³ of dimethylformamide. 2 mmol of N-ethyl-diisopropylamine are added to this solution, followed by 1 mmol of dodecyl isocyanate. The mixture is stirred for 24 hours at 50° C. and the reaction is monitored by HPLC. The urea obtained is filtered off, under cold conditions, in the form of a precipitate due to the excess isocyanate introduced. Acetic acid is then added to pH 4 and the solvent is evaporated off.

The crude product obtained is purified by preparative HPLC. The fractions of interest are combined and lyophilized.

0.134 mmol of salified product is obtained, i.e. a yield of 41.9%.

HPLC: Rt=11.34 min.

¹H NMR spectrum (400 MHz, (CD₃)₂SO-d₆, δ in ppm: 0.89 (t, J=7 Hz: 3H); from 1.25 to 1.45 (mt: 18H); 1.56 (mt: 2H) ; 2.93 (S: 3H) ; 3.15 (mt: 2H) ; from 3.30 to 4.20 (mt: 8H); 7.17 (dd, J=9 and 2 Hz: 1H); 7.22 (d, J=2 Hz: 1H); 7.34 (d, J=8.5 Hz: 2H); from 7.45 to 7.60 (unres. mult.: 1H); 7.63 (d, J=9 Hz: 1H); 7.81 (d, J=8 Hz: 1H); 8.06 (broad d, J=8 Hz: 1H); 8.24 (d, J=8.5 Hz: 2H); 8.43 (broad s: 1H).

### Example 3

### Demonstration of the Formation of Complexes Between Derivative (1) or (2) and DNA by Direct Measurement of Fluorescence

This example illustrates the property of the oligobenzimidazole derivatives according to the invention to form complexes with DNA.

For this, the fluorescence of a mixture of DNA with increasing amounts of derivative (1) or (2) was measured by excitation at 350 nm and detection at 450 nm.

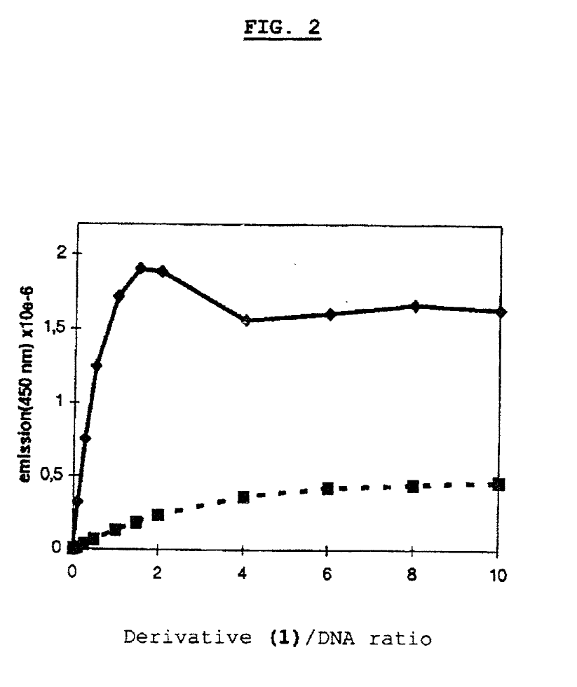

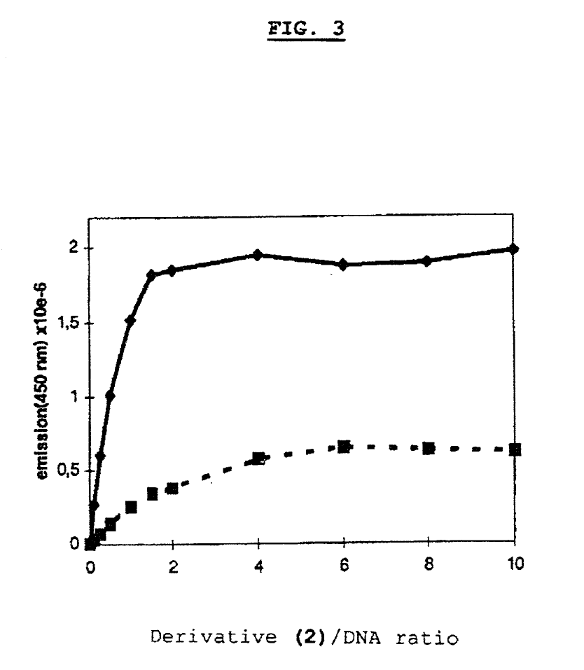

The results are given in FIG. 2 for the formation of complexes with derivative (1) and in FIG. 3 for the formation of complexes with derivative (2).

In all the cases, it is found that when there is no oligobenzimidazole derivative (1) or (2) and when the DNA is in solution alone, no fluorescence is detected. Thereafter, the fluorescence increases with increasing amounts of derivative (1) or (2) until a steady stage is reached. This fluorescence is not identical to the fluorescence emitted by derivative (1) or (2) alone, but, on the other hand, the curves obtained for the DNA/derivative complexes show an emission and excitation spectrum which is similar to that obtained for “Hoechst 33258” (result not shown).

Thus, these results show that derivatives (1) and (2) form complexes with DNA and that the saturation of the DNA groove (concentration of derivative relative to the amount of DNA beyond which no further complex forms) is at an oligobenzimidazole derivative/DNA ratio of about 1.5-2 nmol/μg.

### Example 4

### Electrophoretic Study of an Agarose Gel and Comparison with a Cationic Lipid of the Prior Art

This example complements Example 3 since it demonstrates the formation of DNA/oligobenzimidazole derivative complexes according to the invention. In addition, this example illustrates the specific properties of these DNA/oligobenzimidazole derivative complexes compared with the DNA/cationic lipid complexes of the prior art.

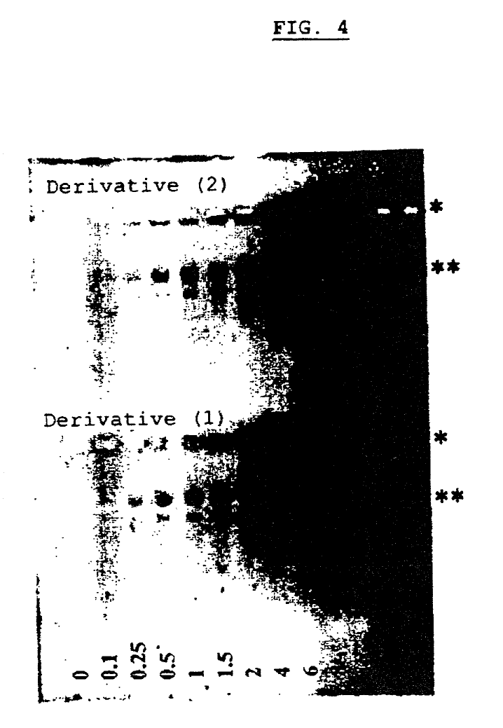

For this, an agarose gel of a DNA plasmid mixed with increasing amounts of derivative (1) or (2) according to the invention was prepared (see FIG. 4). This gel was directly observed under the light of a UV lamp, without being revealed. Two bands could thus be observed by virtue of the specific spectral absorption properties of the derivatives according to the present invention:

a yellow band characteristic of the derivative alone, i.e. not complexed with DNA (band labeled with a “*”): it is observed that the derivative remains at the point of injection,

a blue band characteristic of the derivative complexed with DNA (band labeled with a “**”)

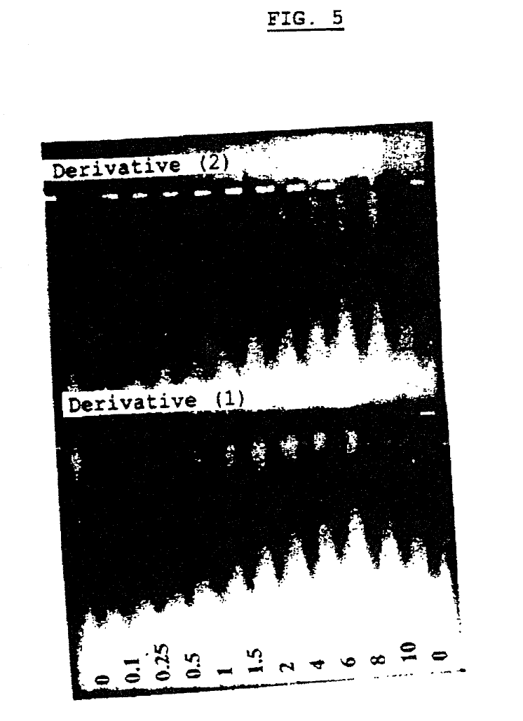

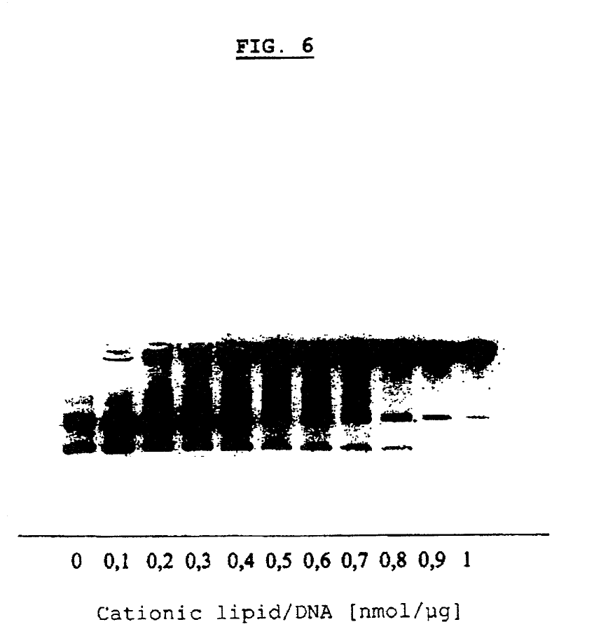

The same agarose gel was then revealed with ethidium bromide (see FIG. 5). It is observed that the DNA migrates in an identical manner to the naked DNA, irrespective of the concentration of derivative 20 according to the invention. This example thus illustrates the fact that the derivatives (1) and (2) give complexes with DNA which have the same electrophoretic mobility properties as the naked DNA, whereas this is not the case when the DNA is complexed with conventional cationic lipids. Specifically, FIG. 6 shows an agarose gel prepared with complexes containing increasing amounts of a cationic lipid: a migration of the complexes formed is observed, which varies with the amount of cationic lipid present with the DNA. This result indicates that the larger the amount of cationic lipid, the more the DNA is compacted and the less it migrates on the gel.

Thus, the oligobenzimidazole derivatives according to the present invention are DNA complexing agents which do not compact DNA, unlike the cationic lipids conventionally used for nonviral gene transfection. The mobility properties of the DNA are thus conserved, even when large amounts of derivatives according to the present invention are added to the DNA to form complexes. This property is particularly advantageous in the aspect of nonviral gene transfection, since it would thus be possible, by virtue of the oligobenzimidazole derivatives according to the present invention, to form complexes with DNA allowing said DNA to be protected against endonucleases without, however, modifying its mobility properties.

In addition, it is thus also possible to form complexes which can be detected by direct methods, in particular without revelation with ethidium bromide, by virtue of the specific fluorescence properties of the derivatives according to the present invention.

### Example 5

### In Vitro Transfection of Genetic Material Complexed with Derivative (2) According to the Invention in the Presence and Absence of Serum

A. Genetic Material Used

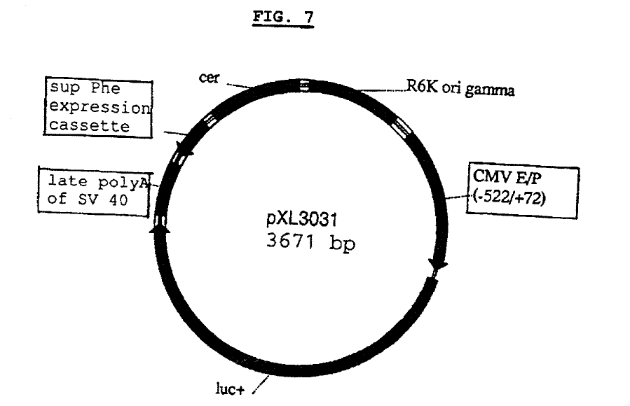

The plasmid used The DNA used is the plasmid pXL3031 (see FIG. 7) as a solution in a mixture of 5% dextrose and 10 mM sodium chloride at a concentration of 0.5 mg/ml or 1.0 mg/ml. This plasmid contains the luc gene encoding luciferase under the control of the cytomegalovirus P/E CMV promoter. Its size is 3671 bp. The plasmid pXL3031 was purified according to the methods described in patent application WO 97/35002.

The nucleic acid solutions are diluted to 20 μg/ml in physiological saline (0.15 M sodium chloride).

B. Cytofecting Solutions (Prepared at the Time of Use)

The oligobenzimidazole derivative (2) according to the invention is dissolved in water to a concentration ranging from 40 to 160 μmol and mixed, volume for volume, with the DNA solution. The final saline concentration is 75 mmol.

C. Transfection

HeLa cells are cultured under suitable conditions on 24-well microplates (2 cm²/well) and are transfected while they are in the exponential growth phase and at 50-70% of confluence.

The cells are washed with twice 0.5 cm³ of medium free of seric proteins and are regrown either in serum-free medium (transfection in the absence of serum) or in whole medium (transfection in the presence of serum). 0.05 cm³ of cytofecting mixture (0.5 μg of DNA/well) are added to the cells (3 wells/DNA-vector condition). When the cells are transfected in the absence of serum, the growth medium is supplemented, 2 hours after the transfection, with a suitable amount of serum.

The transfecting efficacy is evaluated 48 hours after transfection by measuring the expression of luciferase according to the recommendations given for using the Promega kit (“Luciferase Assay System”). The toxicity of the cytofecting mixtures is estimated by measuring the protein concentrations in cell lysates.

The in vitro luciferase activity results relative to the proteins expressed in RLU/5 μl/10 s/μg of protein (written more simply as RLU/μg of protein, “RLU” meaning “relative light unit”) are given in the table below:

| Concentration | | | | | |
|---|---|---|---|---|---|
| (nmol/μg of DNA) | 0 | 2 | 4 | 8 | 10 |
| Derivative (2)/DNA | nd | nd | 1.3 E+03 | 1.7 E+03 | 1.7 E+03 |
| Derivative (2)/ | nd | nd | 9.8 E+02 | 4.6 E+02 | 1.1 E+02 |
| DNA + serum | | | | | |

“nd”means “expression not detectable”.

The results obtained indicate that it is possible to obtain an expression after transfer of genetic material complexed with derivatives according to the present invention in cells in vitro, whether this is in a medium with or without serum.

### Example 6

### In Vivo Transfection of Genetic Material Complexed With Derivative (1) According to the Invention With or Without Electrotransfer

The plasmid used is the same as the one described above for Example 5 (pXL3031). Similarly, the cytofecting solutions are prepared in the same way as in Example 5.

Transfection

25 μl of solutions of derivative (1)/DNA complexes are injected intramuscularly to C57B16 mice, at a rate of 4 μg of DNA/mouse muscle.

The transfection efficacy is evaluated 7 days 5 after transfection by measuring the expression of luciferase according to the recommendations given for using the Promega kit (Luciferase Assay System). The muscles are ground in 1.5 ml of lysis buffer (with protease inhibitors). After assaying the luciferase activity on 10 μl, the results are expressed as RLU/10 μl/10 sec.

The in vivo luciferase activity results in mouse muscles, expressed in RLU/10 μl/10 sec, are collated in the table below:

| Concentration of | | | | |
|---|---|---|---|---|
| derivative (1) in | | | | |
| nmol/μg of DNA) | 0 | 0.2 | 0.5 | 1 |
| In vivo transfection | 9.25 E+04 | 3.45 E+04 | 1.67 E+04 | 1.06 E+04 |
| without electrotransfer | | | | |
| In vivo transfection with | 2.96 E+07 | 1.14 E+07 | 2.19 E+07 | 1.82 E+07 |
| electrotransfer | | | | |

The results obtained indicate that it is possible to obtain an in vivo expression in the muscle after transfer of genetic material complexed to 20 derivatives according to the present invention, whether or not the electrotransfer technique as described in patent applications WO 99/011576 and WO 99/01158 is used.

The present invention is not to be limited in scope b the specific embodiments described herein. Indeed, various modifications of the invention in addition to those described herein will become apparent to those skilled in the art from the foregoing description and the accompanying figures. Such modifications are intended to fall within the scope of the appended claims.

We claim:

1. Oligobenzimidazole derivatives of general formula (I): 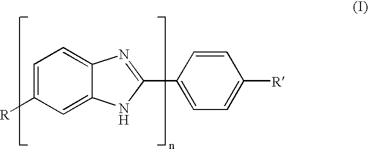
in which
R represents a hydrogen atom, a carboxyl, alkoxycarbonyl, carbamoyl or alkylcarbamoyl radical or a piperazinyl group optionally substituted in position −4 with an alkyl containing 1 to 4 straight or branched carbon atoms, or alternatively R represents an imidazolyl group,
n is an integer equal to 2, 3, 4 or 5, and
R′ represents a group —O—R₃, —S—R₃, NHR₃ or —O—CO—NH—R₃ and R₃ represents an alkyl group,
or alternatively R′ represents a group —NR₄R₅ or —O—CO—NR₄R₅ and R₄ and R₅, which may be identical or different, each represent an alkyl group,
the alkyl radicals mentioned above being, except where specified otherwise, straight or branched, optionally saturated and containing 12 to 22 carbon atoms,
it being understood that R′ is other than OR₃ with R₃ representing a dodecyl substituent when R represents 4-methylpiperazinyl and that n is equal to 2,
as well as the metal salts thereof, the addition salts thereof with the nitrogenous bases and the addition salts thereof with acids.

2. Oligobenzimidazole derivatives according to claim 1, of general formula (II): 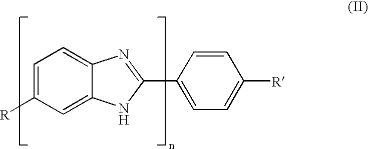
in which
R represents a hydrogen atom or a piperazinyl group optionally substituted in position −4 with an alkyl containing 1 to 4 straight or branched carbon atoms,
n is an integer equal to 2 or 3, and
R′ represents a group —OR₃, NHR₃ or —O—CO—NH—R₃ and R₃ represents an alkyl group,
or alternatively R′ represents a group NR₄R₅ or —O—CO—NR₄R₅ and R₄ and R₅, which may be identical or different, each represent an alkyl group,
the alkyl radicals mentioned above being, except where otherwise specified, straight or branched, optionally saturated and containing 12 to 22 carbon atoms,
it being understood that R′ is other than OR₃ with R₃ representing a dodecyl substituent when R represents 4-methylpiperazinyl and that n is equal to 2,
as well as the metal salts thereof, the addition salts thereof with the nitrogenous bases and the addition salts thereof with acids.

3. Oligobenzimidazole derivatives according to claim 1, characterized in that the derivative is 4-[6-(4-methyl-1-piperazinyl)-1H,3′H-[2,5′]bisbenzimidazol-2′-yl]-1-octadecylcarbamoyloxy phenyl (derivative (1)) or 4-[6-(4-methyl-1-piperazinyl)-1H,3′H- [2,5′]bisbenzimidazol-2′-yl]-1-dodecylcarbamoyloxy phenyl (derivative (2)).

4. Composition, characterized in that it comprises an oligobenzimidazole derivative as defined in claim 1, 2 or 3 or the derivative for which R′ represents a group OR₃ with R₃ representing a dodecyl substituent, R represents 4-methylpiperazinyl and n is equal to 2, and a nucleic acid.

5. Composition according to claim 4, characterized in that it also comprises one or more adjuvants.

6. Compositions according to claim 4, characterized in that it also contains a vehicle which is pharmaceutically acceptable for an injectable or topical formulation or for a formulation in the form of an aerosol.

7. Compositions according to claim 5, characterized in that it also contains a vehicle which is pharmaceutically acceptable for an injectable or topical formulation or for a formulation in the form of an aerosol.

8. Use of an oligobenzimidazole derivative as defined in claim 1, 2 or 3 or of the derivative for which R′ represents a group OR₃ with R₃ representing a dodecyl substituent, R represents 4-methylpiperazinyl and n is equal to 2, for the transfer of nucleic acids into cells in vitro, in vivo or ex vivo.

9. Use of an oligobenzimidazole derivative as defined in claim 1, 2 or 3 or of the derivative for which R′ represents a group OR₃ with R₃ representing a dodecyl substituent, R represents 4-methylpiperazinyl and n is equal to 2, for the preparation of a medicinal product for transferring nucleic acid into cells.

10. Method for transferring nucleic acids into cells, characterized in that it comprises a first step during which the nucleic acid is placed in contact with at least one oligobenzimidazole derivative as defined in claim 1, 2 or 3 or with the derivative for which R′ represents a group OR₃ with R₃ representing a dodecyl substituent, R represents 4-methylpiperazinyl and n is equal to 2, and optionally with one or more adjuvants and/or one or more physiologically compatible vehicles to form a complex, and a second step which consists in placing the complex thus formed in contact with the cells.
